(19) United States
(12) Patent Application Publication
(10) Pub. No.: US 2008/0133016 A1
(43) Pub. Date: Jun. 5, 2008

(54) Spinal arthroplasty device compatible with neural integrity monitoring

(75) Inventor: Eric Steven Heinz, Memphis, TN (US)

Correspondence Address: KRIEG DEVAULT LLP, ONE INDIANA SQUARE, SUITE 2800, INDIANAPOLIS, IN, 46204-2709, US

(73) Assignee: Warsaw Orthopedic, Inc.

(21) Appl. No.: 11/606,539

(22) Filed: Nov. 30, 2006

(51) Int. Cl. A61F 2/44; A61B 17/58; A61B 5/00

(52) U.S. Cl. 623/17.16; 606/99; 600/300

(57) ABSTRACT

A prosthetic implant is disclosed that includes at least one electrode mounted on an outside surface of the prosthetic implant and a connection terminal electrically connected with the electrode. A surgical instrument having a gripping member is used to selectively grip the prosthetic implant. The gripping member includes at least one connector oriented to be connected with the connection terminal of the prosthetic implant when the gripping member is secured to the prosthetic implant. An electrical stimulation signal generator is connected with the surgical instrument for providing electrical stimulation signals to the electrodes of the prosthetic implant thereby allowing a surgeon to neurologically monitor the implantation procedure.

### TECHNICAL FIELD

The present invention relates generally to the field of surgical instrumentation and methods, and more particularly relates to instrumentation and methods for using neural integrity monitoring in conjunction with implantation of an arthroplasty device during a surgical procedure.

### BACKGROUND

In the treatment of diseases, injuries, or malformations affecting spinal motion segments, and especially those affecting the disc tissue, it has long been known to remove some or all of a degenerated, ruptured, or otherwise failing disc. In cases involving intervertebral disc tissue that has been removed or is otherwise absent from a spinal motion segment, corrective measures are taken to ensure the proper spacing of the vertebrae formerly separated by the removed disc tissue. Prosthetic devices may be inserted into the disc space to maintain the structural integrity of the spinal column.

A concern of surgeons performing intervertebral operations is that of inadvertently coming into contact with nerves, including the exiting nerve roots, traversing nerves and the nerves of the caudal equina. The exact location of these nerves is not known prior to surgery. Intervertebral spaces in the spine have other sensitive nerves disposed at locations which are not entirely predictable prior to insertion of the surgical tool and prosthetic device into the intervertebral area. Accordingly, the danger of pinching or damaging spinal nerves when accessing an intervertebral space has proven to be quite limiting to the methods and devices used during spinal surgery. Thus, there remains a need for improved devices and methods for inserting prosthetic devices into place within a patient while not pinching or damaging nerves that may be located in the surgical area.

### SUMMARY

An arthroplasty implant is disclosed that includes an electrode through which an electric stimulation signal is transmitted while the implant is being positioned in a bone structure of a patient. The implant includes a first member having at least an upper surface, a longitudinal side surface, and a lower surface. A first electrode is positioned or located on an outside portion of the longitudinal side surface of the first member and is oriented in an insertion direction of the implant. A connection terminal is housed within the first member and electrically connected with the first electrode. The electrode is oriented such that it faces a direction where, when inserted into a portion of the body, nerves may be located in the portion of the body. The electrode and the connection terminal may be removed from the implant once the implant is properly positioned within the patient.

In another representative embodiment, the implant includes a second member that also includes an upper surface, a longitudinal side surface, and a lower surface. The second member also preferentially includes a second electrode that is oriented on the longitudinal side surface of the second member in an insertion direction of the implant. A connection terminal is positioned within the second member and is electrically connected with the electrode of the second member. In some embodiments, the second member is removably pivotally connected with the first member such that it may rotate about a predetermined axis.

Another aspect of the present invention discloses an arthroplasty system that is capable of detecting nerves that may come into contact with or are within close proximity with an implant while being positioned in the body. The system includes a prosthetic implant having an electrode mounted on an outside surface of the prosthetic implant and a connection terminal connected with the electrode. The system further includes a surgical instrument that has a gripping member operable to selectively grip the prosthetic implant. The gripping member includes at least one electrical connector that is oriented to be connected with the connection terminal of the prosthetic implant when the gripping member is secured to the prosthetic implant. As such, the gripping member is capable of making an electrical connection with the prosthetic implant. The system also includes an electrical stimulation signal generator that is connected with the surgical instrument for providing electrical stimulation signals to the electrodes through the surgical instrument.

Yet another aspect of the present invention discloses a method of installing a prosthetic implant into a bone structure of a patient while at the same time allowing a surgeon to neurologically monitor the surgical area to determine if the prosthetic implant is in contact with, or in close proximity to, nerves that may be located in the surgical area. The surgical method includes creating a window to a bone structure in a body. Once the window has been created, at least a portion of the bone structure is removed using one of various techniques known in the art. A prosthetic implant is provided that includes at least one electrode positioned on an outside surface of the prosthetic implant, wherein the electrode is connected with at least one connection terminal positioned in the prosthetic implant. The prosthetic implant is removably secured to a surgical instrument such that a connector of the surgical instrument is electrically connected with the connection terminal of the prosthetic implant. The prosthetic implant is then inserted through the window to replace the portion of the bone structure that has been removed using the surgical instrument. As the prosthetic implant is inserted, an electrical stimulation signal is generated that is selectively provided to the electrode while the prosthetic implant is being inserted in the window to replace the portion of the bone structure that has been removed.

Other systems, methods, features and advantages of the invention will be, or will become apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The components in the figures are not necessarily to scale, emphasis instead being placed upon illustrating the principles of the invention. Moreover, in the figures, like reference numerals designate corresponding parts throughout the different views.

FIG. 1 illustrates a representative spinal column.

FIG. 2 is a cross-sectional view of a representative vertebrae and associated vertebral disc of the spinal column.

FIG. 3 illustrates a representative spinal segment illustrating nerves of the spinal column.

FIG. 4 illustrates various planes in relation to a human body.

FIG. 5 illustrates a representative spinal segment having a intervertebral disc removed.

FIG. 6 is a perspective view of a representative prosthetic disc implant.

FIG. 7 is another perspective view of the prosthetic disc implant illustrated in FIG. 6.

FIG. 8 is another perspective view of the prosthetic disc implant illustrated in FIG. 6.

FIG. 9 is a perspective view of a surgical instrument for gripping the prosthetic disc implant for insertion into the body.

FIG. 10 is a perspective view of a surgical instrument connected with the prosthetic disc implant.

FIG. 11 is a perspective view of the surgical instrument.

FIG. 12 is a block diagram of an implant system using the surgical instrument and the prosthetic disc implant.

FIG. 13 illustrates another representative prosthetic disc implant positioned between two adjacent vertebrae.

FIG. 14 illustrates yet another representative prosthetic disc implant positioned between two adjacent vertebrae.

FIG. 15 illustrates a representative connector of the surgical instrument.

FIG. 16 is a front view of a respective articulate member illustrating a slit and connection terminals.

### DETAILED DESCRIPTION

The invention relates to implantable devices, including prosthesis suitable for implantation within the body to restore and/or augment connective tissue such as bone, and systems for determining the presence of a nerve coming in contact with the implant device. In various embodiments, the implantable devices can include devices designed to replace missing, removed or resected body parts or structure. The implantable devices, apparatus or mechanisms are configured such that the devices can be formed from parts, elements or components which alone, or in combination, comprise the device. The implantable devices can also be configured such that one or more elements or components are formed integrally to achieve a desired physiological, operational or functional result such that the components complete the device. Functional results can include the surgical restoration and functional power of a joint, controlling, limiting or altering the functional power of a joint, and/or eliminating the functional power of a joint by preventing joint motion. Portions of the device can be configured to replace or augment existing anatomy and/or implanted devices, and/or be used in combination with resection or removal of existing anatomical structure.

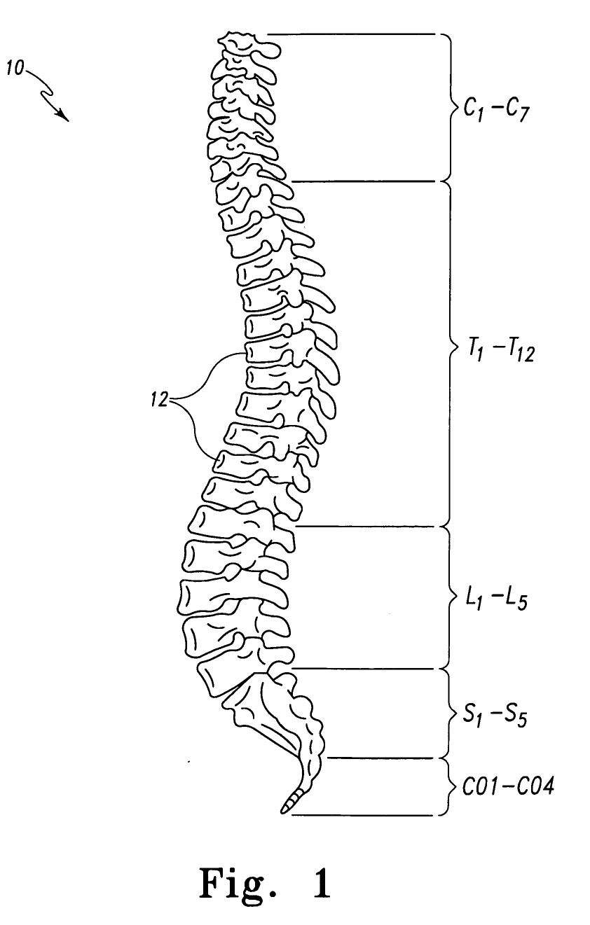

The devices disclosed herein are preferably, but not exclusively, designed to interact with the human spinal column 10, as shown in FIG. 1, which is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4. Although the preferred embodiment is designed to interact with the human spinal column 10, the present invention could also be taken advantage of in conjunction with any implantable device that may be inserted or placed near nerve tissue.

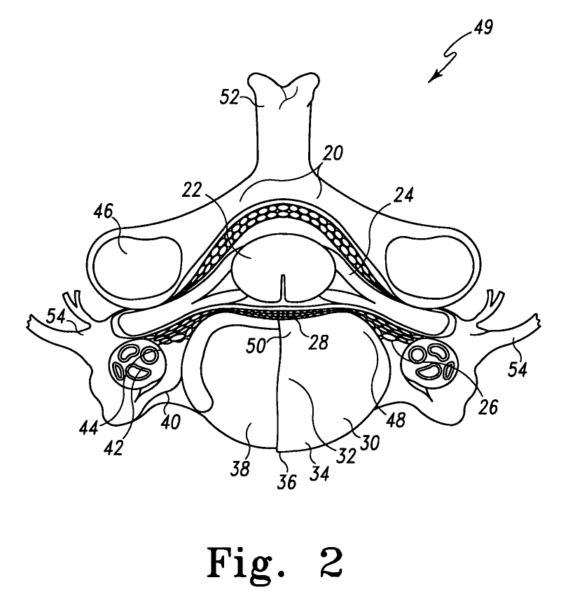

FIG. 2 illustrates a cross-sectional anatomical view of a vertebra 49 and associated intervertebral disc 30. Structures of a typical cervical vertebra 49 (superior aspect) are shown in FIG. 2 and include a lamina 20, a spinal cord 22, a dorsal root of spinal nerve 24, a ventral root of spinal nerve 26, a posterior longitudinal ligament 28, a intervertebral disc 30, a nucleus pulposus 32, a annulus fibrosus 34, a anterior longitudinal ligament 36, a vertebral body 38, a pedicle 40, a vertebral artery 42, vertebral veins 44, a superior articular facet 46, a posterior lateral portion of the annulus 48, a posterior medial portion of the annulus 50, a spinous process 52, and a spinal nerve 54. In FIG. 2, one side of the intervertebral disc 30 is not shown so that the anterior vertebral body 38 can be seen. As clearly set forth in FIG. 2, detection of nerves when implanting devices is important because the spinal column 10 and areas surrounding the spinal column 10 contain numerous nerves.

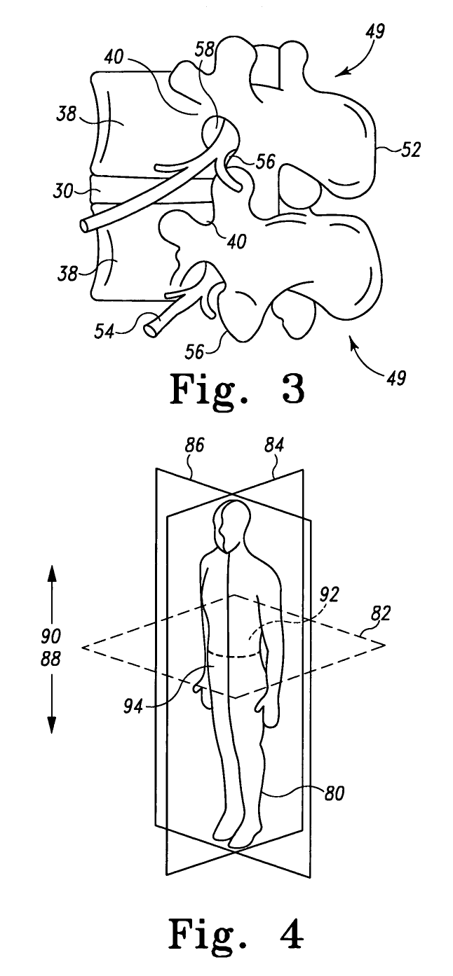

FIG. 3 provides a posterior-lateral anatomical view of two representative vertebrae 49. An inferior articular process 56, along with the pedicle 40 and the spinal nerve 54, form a small “triangular” window through which introduction of an instrument can be achieved from the posterior lateral approach. As illustrated, the spinal nerve 54 protrudes outwardly from an intervertebral foramen 58 formed between respective adjacent vertebrae 12. As such, when an intervertebral disc 30 is removed, a space is left between the respective vertebrae 49. However, as illustrated in FIG. 2, the area surrounding intervertebral disc 30 is filled with nerves. The intervertebral disc 30 has the same general shape as the vertebral body 38 of the vertebrae 49.

Referring to FIG. 4, in order to understand the configurability, adaptability, and operational aspects of the invention, it is helpful to understand the anatomical references of the body 80 with respect to which the position and operation of the devices, and components thereof, are described. There are three anatomical planes generally used in anatomy to describe the human body and structure within the human body: the axial plane 82, the sagittal plane 84 and the coronal plane 86. Additionally, devices and the operation of devices are better understood with respect to the caudal 88 direction and/or the cephalic direction 90. Devices positioned within the body 80 can be positioned dorsally 92 (or posteriorly) such that the placement or operation of the device is toward the back or rear of the body. Alternatively, devices can be positioned ventrally 94 (or anteriorly) such that the placement or operation of the device is toward the front of the body. Various embodiments of the spinal devices and systems of the present invention may be configurable and variable with respect to a single anatomical plane or with respect to two or more anatomical planes. In addition, the devices disclosed herein may be inserted in the body 80 using a posterior approach, an anterior approach or a lateral approach.

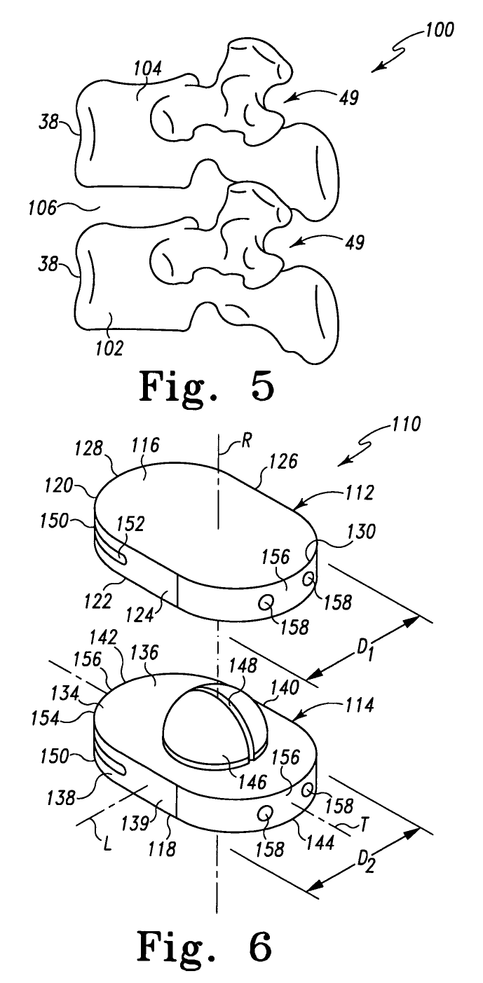

Referring to FIG. 5, for further example, two vertebrae 49 will be discussed, designated as spinal segment 100 including a lower vertebrae 102 and an upper vertebrae 104 each having a vertebral body 38. In one embodiment, some or all of the intervertebral disc 30 that would have been positioned between the two vertebrae 102, 104 is removed via a discectomy or a similar other procedure. Removal of the diseased or degenerated disc 30 results in the formation of an intervertebral space or window 106 between the lower and upper vertebrae 102, 104.

As illustrated in FIGS. 6-8, a representative embodiment of an intervertebral prosthetic disc 110 for insertion into the vertebral space 106 (See FIG. 5) is illustrated. The prosthetic disc 110 provides for articulating motion, thereby restoring motion to the spinal segment 100 defined by the lower and upper vertebrae 102, 104. The prosthetic disc 110 includes a first articular member 112 and a second articular member 114. The articular members 112, 114 cooperate to form the prosthetic disc 110. The prosthetic disc 110 is sized and configured for disposition within the intervertebral space 106 between the lower and upper vertebrae 102, 104. For the purpose of the present invention, it should be noted that the shape of the prosthetic discs disclosed herein are for illustrative purposes only and should not be construed as a limitation of the present invention. It should be appreciated that the invention disclosed herein may be utilized or incorporated into almost any type of prosthetic disc regardless of whether or not the prosthetic disc is designed to be inserted from an anterior approach, a lateral approach, or a posterior approach. One such disc that may be modified to incorporate the present invention may be found in U.S. Patent Application 2006/0030860, which is hereby incorporated by reference in its entirety.

The prosthetic disc 110 provides relative pivotal and rotational movement between the adjacent vertebral bodies 102, 104 to maintain or restore motion substantially similar to the normal bio-mechanical motion provided by a natural intervertebral disc 30. More specifically, the articular members 112, 114 are permitted to pivot relative to one another about a number of axes, including lateral or side-to-side pivotal movement about longitudinal axis L and anterior-posterior pivotal movement about transverse axis T. It should be understood that in one embodiment, the articular members 112, 114 are permitted to pivot relative to one another about any axis that lies in a plane that intersects longitudinal axis L and transverse axis T. Furthermore, the articular members 112, 114 are permitted to rotate relative to one another about a rotational axis R. Although the prosthetic disc 110 has been illustrated and described as providing a specific combination of articulating motion, it should be understood that other combinations of articulating movement are also possible, such as, for example, relative translational or linear motion, and such movement is contemplated as falling within the scope of the present disclosure.

Although the articular members 112, 114 of prosthetic disc 110 may be formed from a wide variety of materials, in one embodiment, the articular components 112, 114 are formed of a cobalt-chrome-molybdenum metallic alloy (ASTM F-799 or F-75). However, in alternative embodiments, the articular members 112, 114 may be formed of other materials such as titanium or stainless steel, a polymeric material such as polyethylene, or any other biocompatible material, alloy or polymer that would be apparent to one of ordinary skill in the art.

The articular members 112, 114 each include a bearing surface 116, 118, respectively, that may be positioned in direct contact with vertebral bone and is preferably coated with a bone-growth promoting substance, such as, for example, a hydroxyapatite coating formed of calcium phosphate. Additionally, the bearing surfaces 116, 118 of the articular components 112, 114, respectively, may be roughened prior to being coated with the bone-growth promoting substance to further enhance bone on-growth. Such surface roughening may be accomplished by way of, for example, acid etching, knurling, application of a bead coating, or other methods of roughening that would be apparent to one of ordinary skill in the art. Other structures, such as a keel (not illustrated), may be included on the bearing surface 116, 118.

Articular member 112 includes a support plate 120 having a lower surface 122 and the bearing surface 116. Support plate 120 is preferentially sized and shaped to provide a technically feasible maximum amount of endplate support for the upper vertebra 104 while minimizing the lateral window associated with insertion of the prosthetic disc 110. For example, the support plate 120 may be shaped such that longitudinal sides 124, 126 of the support plate 30 are substantially parallel and are separated by a distance D1. Additionally, the lateral sides 128, 130 of the support plate 120 may take a curved configuration to correspond to the curvature of the upper vertebra 104.

Referring to FIG. 7, in one embodiment, the articular member 112 includes a recess 132. In one embodiment, the recess 132 has a concave shape, and is configured as a spherical-shaped socket. However, it should be understood that other configurations of the recess 132 are also contemplated, such as, for example, cylindrical, elliptical or other arcuate configurations or possibly non-arcuate configurations. The remaining portion of the articular surface 122 can be angled or otherwise configured to facilitate the insertion and/or use of the prosthetic disc 110.

Articular component 114 includes a support plate 134 having an upper articular surface 136 and the lower bearing surface 138. Support plate 134 is sized and shaped to provide a technically feasible maximum amount of endplate support for the lower vertebra 102 while minimizing the window associated with insertion of the prosthetic disc 110. For example, the support plate 134 may be shaped such that longitudinal sides 139, 140 of the support plate 134 are substantially parallel and are separated by the distance D2. Additionally, the lateral sides 142, 144 of the support plate 134 may take a curved configuration to correspond to the curvature of the endplate of the lower vertebra 102.

The articular member 114 includes a projection 146 having a convex shape, which may be configured as a spherical-shaped ball (half of which is shown). It should be understood that other configurations of the projection 146 are also contemplated, such as, for example, cylindrical, elliptical or other arcuate configurations or possibly non-arcuate configurations. It should also be understood that the remaining portion of articular member 114 may take on planar or non-planar configurations, such as, for example, an angular or conical configuration extending about the projection 146. A surface depression or cavity 148 may be defined along a portion of the projection 146 to provide a means for clearing out matter, such as particulate debris, that is disposed between the abutting articular members 112, 114. In other embodiments, the convex articular surface of the projection 146 may alternatively define a generally smooth, uninterrupted articular surface. In another embodiment, the convex projection 146 and the concave recess 132 may define a surface depression to facilitate removal of particulate matter disposed between the abutting articular members 112, 114.

Referring collectively once again to FIGS. 6-8, an insertion surface portion 150 of the longitudinal sides 124, 126 and 139, 140 of the upper and lower articulate members 112, 114 include at least one electrode 152 located on an outer surface of the longitudinal sides 124, 126 and 139, 140. As illustrated, the electrode 152 wraps around the posterior corners 154 and front 156 of the longitudinal sides 124, 126 and 139, 140 of the articulate members 112, 114. It should be appreciated that the electrodes 152 are positioned on the prosthetic disc 110 such that they face an insertion direction of the prosthetic device 110. As such, the prosthetic disc 110 illustrated in FIGS. 6-8 is inserted into a respective spinal segment 100 (See FIG. 2) such that the electrodes 152 face the location in which nerves may be located in the spinal segment 100. As set forth in greater detail below, the electrodes 152 are used to stimulate any nerves that may be in close proximity to either articulate member 112, 114.

An opposite end of the upper and lower articulate members 112, 114 include connection terminals 158 that are recessed into the articulate members 112, 114. The electrodes 152 of the upper and lower articulate members 112, 114 are electrically connected with the connection terminals 158. In this embodiment, the connection terminals 158 are positioned on an opposing side of the lower and upper articulate members 112, 114 as it relates to the electrodes 152. In alternative embodiments, the connection terminals 158 and electrodes 152 may be positioned in any arrangement convenient for the monitoring of nerves and ease of hook-up. The connection terminals 158 allow electrical stimulations to be generated by the electrodes 152 that stimulate nerves that are near the electrodes 152 and as such, are near the prosthetic disc 110. As set forth below, an electric signal is provided to the electrodes 152 from a signal generator through the connection terminals 158.

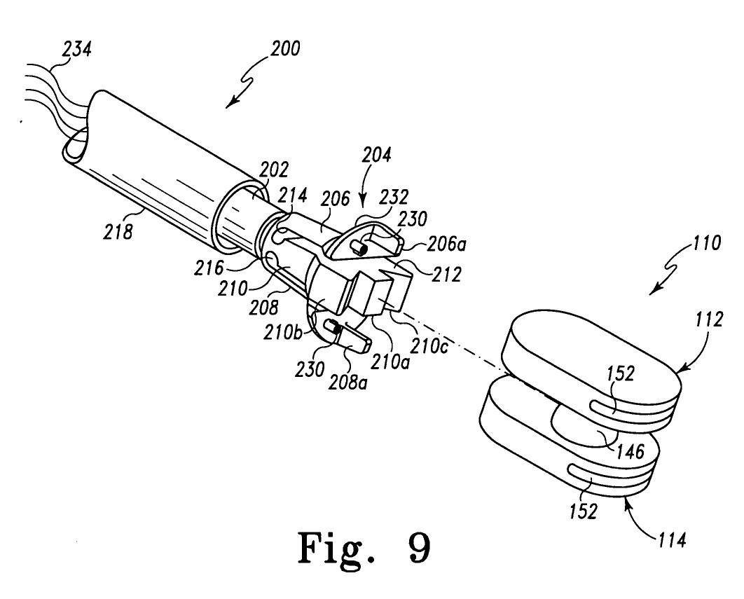

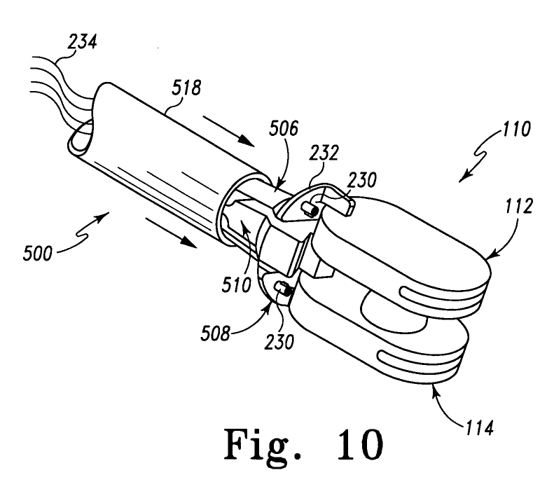

Referring to FIGS. 9-11, shown therein is one embodiment of a representative surgical instrument 200 used to insert the prosthetic disc 110 into the intervertebral space or window 106 (See FIG. 2). The surgical instrument 200 includes an elongated body or rod 202 having a proximal end and a distal end. A gripping device 204 is connected to the distal end of the rod 202. The gripping device 204 is designed to engage the prosthetic disc 110. The gripping device 204 includes an upper body 206, a lower body 208, and a middle body 210. The upper body 206 is adapted for engaging the bearing surface 116 of the first member 112 of the prosthetic disc 110. The lower body 208 is adapted for engaging the bearing surface of the second member 114 of the prosthetic disc 110. It is to be understood that the descriptive labels upper, lower, and middle are in no way intended to the limit the positioning of the components in this disclosure. Rather the use of the labels is a matter of convenience for describing the embodiment.

The middle body 210 includes an upper surface 212 and a lower surface (not shown, but located on the opposing side of the middle body from the upper surface) designed to engage the articular surfaces of the first and second members 112, 114, respectively. The upper and lower surfaces may be shaped and adapted for engaging notches, indentions, projections, recesses, apertures, or other contours of the first and second members 112, 114 of the prosthetic disc 110. Again the labels upper and lower are not intended to limit the positioning the surfaces. The use of labels is simply a matter of convenience.

The middle body 210 may be shaped to maintain a predetermined distance between the first and second members 112, 114. In one embodiment, the middle body 210 has a height representing the desired distance of separation between the first and second components 112, 114. It is fully contemplated that the middle body 210 may have varying heights in a single embodiment to accommodate the shape and contours of the first and second members 112, 114. Further, the separation created by the middle body 210 may be designed to accommodate the insertion of a intervertebral disc prosthesis between vertebral bodies in a spondylosed relationship.

In one embodiment, the middle body 210 includes an engagement profile 210a. The engagement profile 210a may be shaped to mate with any indentions, notches, channels, or other contours of the articular surfaces of the first and second members 112, 114. In this respect, the engagement profile 510a is designed to help securely engage the prosthetic disc 110 while maintaining the desired space between the first and second members 112, 114. It is fully contemplated that the engagement profile 210a may be of any shape and have surface features designed to prevent unwanted rotation and movement of the prosthetic disc 110.

The middle body 210 may include guiding profiles 210b, 210c to prevent unwanted rotation or movement of the first and second members 112, 114 during insertion and manipulation. The guiding profiles 210b, 210c may be shaped to conform to the contours of the first and second components 112, 114 of the prosthetic disc 110. The guiding profiles 210b, 210c may be used to help maintain the predetermined space between the first and second members 112, 114. However, it should be understood that the guiding profiles 210b, 210c may be used only for preventing unwanted rotation and movement of the prosthetic disc 110 during insertion and still be within the present disclosure.

The surgical instrument 200 includes two pivot points 214, 216 that allow the upper and lower bodies 206, 208 to move in respect to the middle body 210 for selective engagement of the first and second components 112, 114. The pivot points 214, 216 allow a dispersion of forces in a manner that prevents the components of gripping member 204 from breaking without compromising the ability to move. The pivot points 214, 216 may be replaced by a fulcrum or by other movable joints or mechanisms to allow for selective engagement of the prosthetic disc 110. It should be understood that such alternatives to the pivot points 214, 216 are fully contemplated as falling within the scope of the present disclosure.

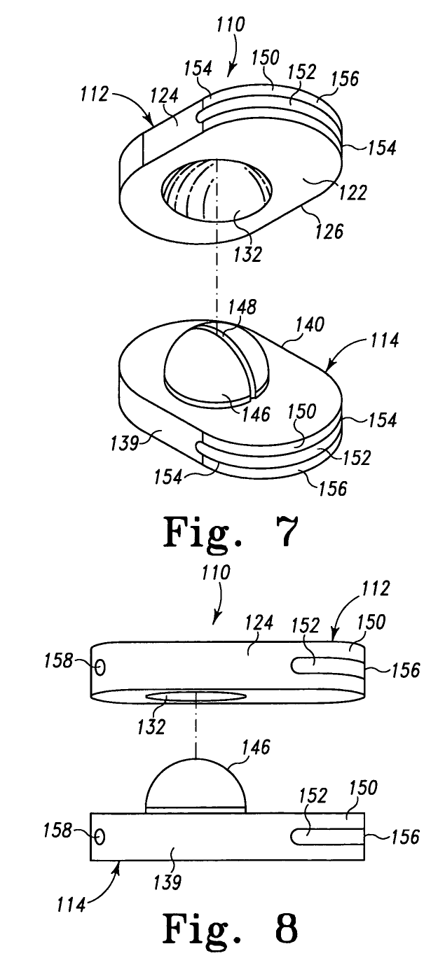

The surgical instrument 200 includes a compression sleeve 218 for selectively moving the upper and lower bodies 206, 208 with respect to the middle body 210 to engage the prosthetic disc 110. The compression sleeve 218 fits over the rod 202 so that it can slide from a disengaged position (See FIG. 8) to an engaged position (See FIG. 9), and vice-versa. The surgical instrument 200 may include or be adapted to use a mechanism other than a compression sleeve for engaging the prosthetic disc 110, for example, a hemostat, and such alternatives are within the present disclosure.

There are several ways the compression sleeve 218 may be manipulated to selectively engage the prosthetic disc 110. In one embodiment the compression sleeve 218 has a length extending along the length of the rod 202 such that a surgeon or other person using the surgical instrument can move the compression sleeve directly, even after insertion. That is, moving the compression sleeve 218 may be accomplished by pushing or pulling on the compression sleeve itself. Moving the compression sleeve 218 then may be used to engage or disengage the prosthetic disc 110. The compression sleeve 218 could be attached to or adapted to interface with a mechanism (not shown) to allow the person using the surgical instrument 200 to engage or disengage the compression sleeve via the mechanism and not the compression sleeve directly. In such an embodiment the mechanism would be accessible to the person using the surgical instrument 200 even after insertion so that the surgical instrument could be disengaged from the prosthetic disc 110.

In another embodiment the compression sleeve 218 may be threaded to the rod 202 such that rotating the compression sleeve about its transverse axis T will move the compression sleeve up or down the rod to engage or disengage the prosthetic disc 110, depending on the direction of rotation. Threading the compression sleeve 218 to the rod 202 provides advantages similar to using a locking mechanism or biasing the surgical instrument 200 to an engaged position. That is, threading the compression sleeve 218 can prevent the surgeon from having to concentrate on keeping the prosthetic disc 110 engaged during insertion and also allows free movement of the hand or hands that may be required to keep the surgical instrument 200 in an engaged position. This, in turn, facilitates more precise insertion of the prosthetic disc 110. In continuation, it should be noted that it is fully contemplated that the surgical instrument 200 may be biased to an engaged position or include a locking mechanism (not shown), including embodiments where the compression sleeve 218 is threaded.

The surgical instrument 200 may also include a plurality of electrical connectors 230 that protrude outwardly from a forward facing surface 232 of the gripping device 204 of the surgical instrument 200. The connectors 230 are arranged or positioned on the surface 232 such that they are in alignment with and may be connected to the connection terminals 158 of the prosthetic disc 110. In one embodiment, the connectors 230 are capable of being removably connected to the connection terminals 158. In an alternative embodiment, the connectors 230, once connected to the connection terminals 158 of the prosthetic disc 110, cannot be unplugged from the connection terminals 158. As set forth in greater detail below, this allows the surgical instrument 200 to be used to pull the electrodes 152 and connection terminals 158 out of the prosthetic disc 110 once properly positioned in a patient. The connectors 230 are connected to a plurality of electrically conductive wires 234 that run inside the surgical instrument 200. In alternative embodiments, the wires 234 may be positioned outside of the surgical instrument 200.

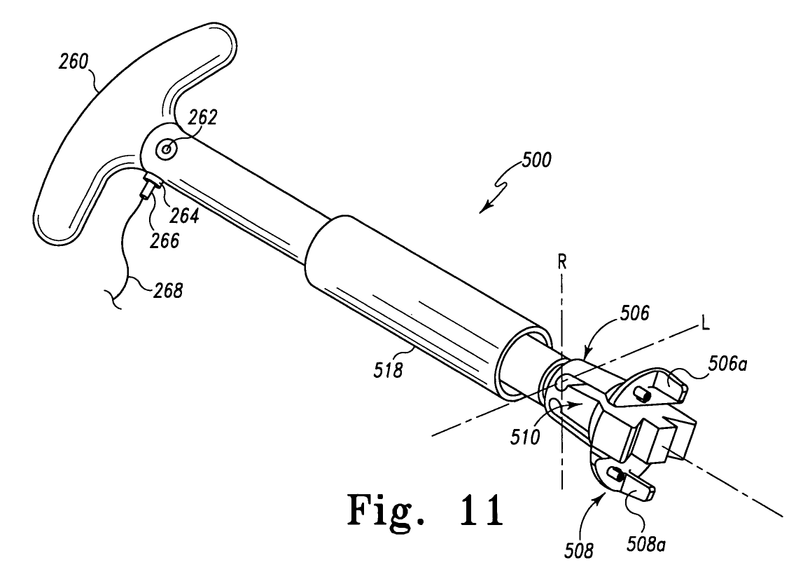

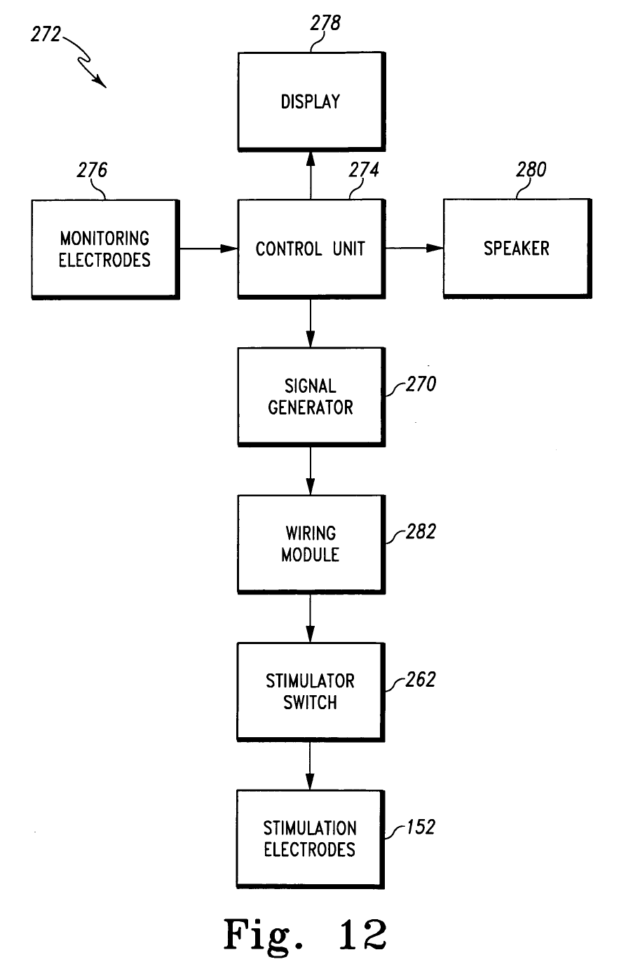

Referring to FIG. 11, the surgical instrument 200 has a handle portion 260 that includes a stimulator switch 262. The wires 234 of the connectors 230 are electrically connected with the stimulator switch 262. The surgical instrument 200 may also include a stimulation input port 264 that is electrically connected with the stimulator switch 262. A plug-in 266 may be inserted into the stimulation input port 264. The plug-in 266 may include an electrically conductive wire 268 that is connected with a stimulation signal generator 270 (See FIG. 12).

Referring to FIG. 12, another aspect of the present invention discloses a nerve detection system 272 for use during surgical procedures in which a prosthetic implant, such as prosthetic disc 110, is being placed in a portion of the body 80 that may be surrounded by nerves. The system 272 uses electromyography (“EMG”) to detect the presence of nerves, such as spinal nerve 54, during the surgical procedure. As set forth above, the inadvertent contact of nerves during surgical procedures may cause complications after surgery. As such, it is desirable to avoid contacting nerves during arthroplasty procedures.

As known in the art, EMG measures the electrical discharges produced in muscles. In a typical EMG application, a thin needle is inserted into the muscle that is being studied. An instrument records the electrical activity in the muscle at rest and as it is contracted. The size, duration and frequency of the signals that are recorded help determine if there is damage to the muscle or to the nerves leading to the muscle. Another typical EMG application is a nerve conduction study. In this study, electrodes are placed on the skin of a patient over a nerve that is to be studied. A small electrical impulse is passed through one electrode. An instrument measures the resulting electrical activity, such as the time it takes for the impulse to travel to another electrode. In some nerve disorders, the speed of the signal is dramatically slowed.

In the present invention, EMG is used to detect when and if the prosthetic disc 110 is about to come into contact with a respective nerve or is in contact with a nerve, such as spinal nerve 54, for example. The system 272 includes a control unit 274 that comprises a microprocessor based control unit that may include a processor, memory, input and output ports, an analog to digital converter and a digital to analog converter to name a few. The signal generator 270 may be connected with and controlled by the control unit 274. The signal generator 270 is used to generate a controlled stimulation signal that is selectively passed to the stimulation electrodes 152 of the prosthetic disc 110. In alternative embodiments, a digital to analog converter of the control unit 274 may be used to generate the controlled stimulation signal. In addition, a wiring module 282 may be included that may connect the stimulator switch 262 to the signal generator 272. The wire 268 (See FIG. 1) from plug-in 266 may connect the stimulator switch 262 with the signal generator 272 via the wiring module 282. The wiring module 282 allows more than one stimulation electrode 152 to receive signals generated by the signal generator 270.

The system 272 may also include a plurality of monitoring electrodes 276 that are connected with the control unit 274. The monitoring electrodes 276 are positioned in or near various muscle or nerve locations throughout the body 80 of the patient. In one embodiment, as many as twenty (20) monitoring electrodes 276 may be placed in or on the body of the patient near muscle or nerve locations associated with the area undergoing surgery. The controlled stimulation signal may be generated as a pulse signal and when the stimulation electrodes 152 approach a nerve, such as spinal nerve 54, it causes the nerve to generate a reaction in the form of an electric signal that may be picked up or detected by one or more of the respective monitoring electrodes 276.

The system 272 is used by surgeons to locate and identify nerves during spinal surgery. The system 272 provides intuitive audible and visual real-time feedback to aid in decision-making during surgery. Surgeons can monitor the spinal cord and nerve roots throughout every stage of inserting the prosthetic disc 110 into the spinal segment 100 of the patient. The system 272 allows surgeons to optimize the placement of the prosthetic disc 110 while at the same time substantially reduces the risk of nerve root irritation or damage.

To use the system 272, monitoring electrodes 276 are placed in muscles of associated nerve roots or on the skin above the respective muscle. If the nerve root becomes irritated or excited by one of the stimulation signals generated by the stimulation electrodes 152 during implantation of the prosthetic disc 110, an electric signal is generated by the nerve root that is detectable by one or more respective monitoring electrodes 276. The control unit 274 may then process this signal and generate a visible illustration on a display 278 and/or an audible warning through a speaker 280. The system 272 provides advance warning to the surgeon of contact with a nerve so that the surgeon may prevent irritation or nerve damage.

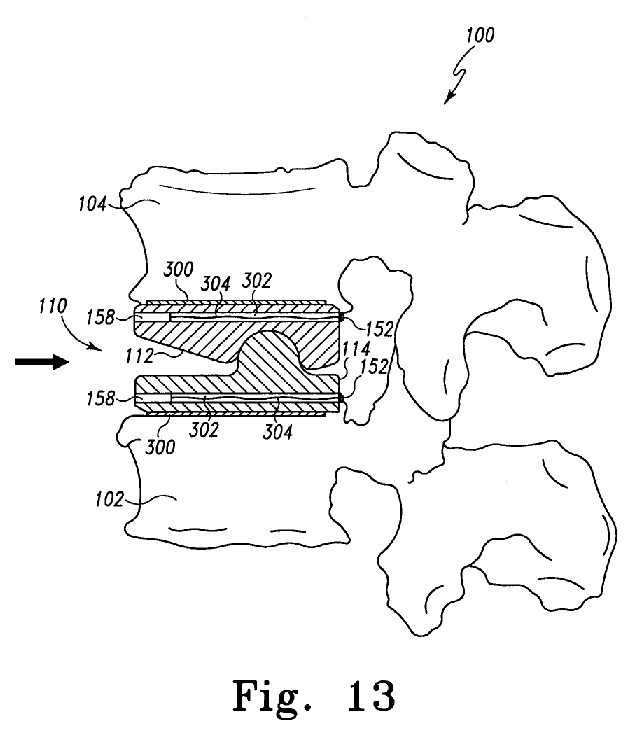

Referring to FIG. 13, a cross-sectional view of another representative prosthetic disc 110 is illustrated properly positioned in the spinal segment 100 between adjacent vertebrae 102, 104. The bearing surfaces 116, 118, respectively, are positioned in direct contact with vertebral bone and are coated with a bone-growth promoting material 300. The upper and lower articulate members 112, 114 include an aperture 302 that runs from a distal end to a proximal end of the upper and lower articulate members 112, 114. The stimulating electrode 152 includes an insulated wire 304 that is connected with the connection terminals 158 housed within the aperture 302. The connection terminals 158 may be friction fit into the aperture 302 or a biocompatible adhesive may be used. The connection terminals 158 provide an electrical connection, via wire 304, to the stimulation electrodes 152.

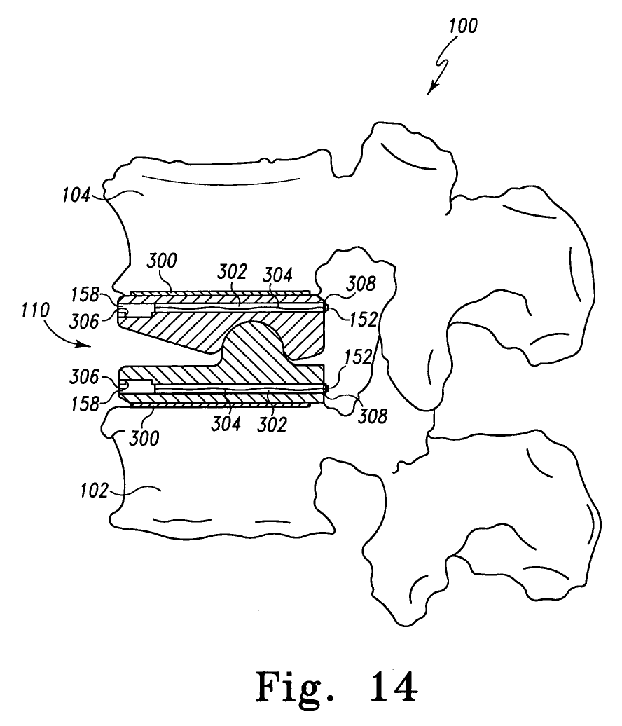

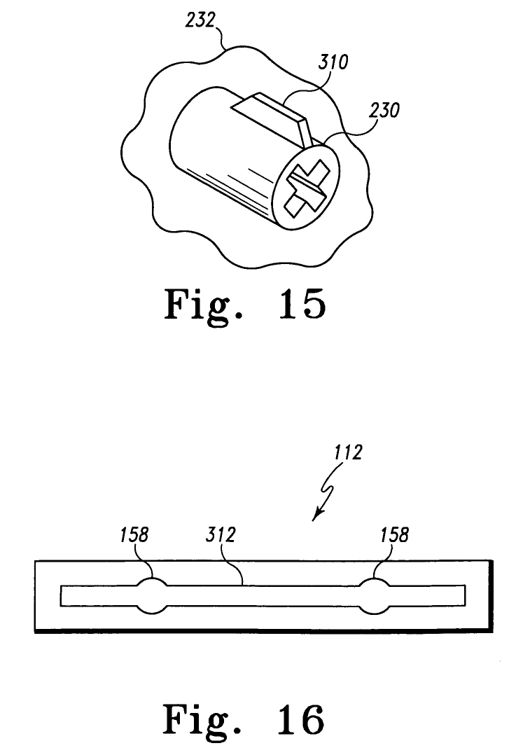

Referring to FIGS. 14 and 15, in alternative embodiments of the present invention, the connector 230 of the surgical instrument 200 may include a locking member 310. The locking member 310 is spring biased to be maintained in an outwardly protruding position. However, the locking member 310 is capable of being depressed into a retracted position so that the connector 230 may be positioned within and connected with a respective connection terminal 158 of the prosthetic disc 110. As illustrated in FIG. 14, an inner portion of the connection terminal 158 includes a locking recess 306 that receives the locking member 310 of the connector 230. As such, once the connectors 230 are connected with the connection terminals 158 of the prosthetic disc 110, the locking member 310, being spring biased to maintain itself in an outward position, locks the connectors 230 into place in the connection terminals 158 of the prosthetic disc 110.

Referring collectively to FIGS. 14 and 16, the electrodes 152 may be connected with the upper and lower articulate members 112, 114 via a flexible retention member 308. Once the prosthetic disc 110 is in proper position, the surgeon may release the prosthetic disc 110 from the surgical instrument 200. After the prosthetic disc 110 is no longer being gripped by the surgical instrument 200, the surgical instrument 200 may be gently retracted or pulled away from the surgical area. Pulling back on the surgical instrument 200 causes the connection terminals 158, the insulated wire 304 and the electrodes 152 to be removed from the prosthetic disc 110.

As previously set forth, the connection terminals 158 may be friction fit inside the articulate members 112, 114 such that application of a predetermined amount of force in a direction away from the prosthetic disc 110 causes the connection terminals 158 to release from the articulate members 112, 114. Since the connection terminals are connected with the electrodes 152 via insulated wire 304, this also causes the electrodes 152 to be pulled through the aperture 302 and out of the prosthetic device 110. As illustrated in FIG. 16, aperture 302 may include a slit 312 that runs substantially through the articulate member 112 that provides enough space for the electrodes 152 to be pulled through and out of the prosthetic disc 110. Although not specifically illustrated, it should be appreciated that the second articulate member 114 may also include a similar slit 312.

While the invention has been illustrated and described in detail in the drawings and foregoing description, the same is to be considered as illustrative and not restrictive in character.

What is claimed is:

1. An arthroplasty implant, comprising:
a first member having an upper surface, a longitudinal side surface, and a lower surface;
a first electrode positioned on an outside portion of said longitudinal side surface of said first member oriented in an insertion direction of said arthroplasty implant; and
a connection terminal housed within said first member and electrically connected with said first electrode.

2. The arthroplasty implant of claim 1, further comprising a second member having a second upper surface, a second longitudinal side surface, and an upper surface.

3. The arthroplasty implant of claim 2, wherein said second member includes a second electrode positioned on an outside portion of said longitudinal side surface and a connection terminal electrically connected with said second electrode.

4. The arthroplasty implant of claim 3, wherein said second member includes a projection protruding upwardly from said upper surface.

5. The arthroplasty implant of claim 4, wherein said first member includes a recess in said lower surface, wherein said projection of said second member is pivotally positioned in said recess of said first member.

6. The arthroplasty implant of claim 1, wherein said first electrode is connected with said connection terminal with an insulated wire positioned within an aperture located inside said first member.

7. The arthroplasty implant of claim 6, wherein said electrode, said insulated wire, and said connection terminal are removably positioned in said first member through said aperture.

8. The arthroplasty implant of claim 1, wherein said member is formed from a biocompatible polymer.

9. The arthroplasty implant of claim 1, wherein said first member is formed from a metallic material.

10. An arthroplasty system, comprising:
a prosthetic implant having an electrode mounted on an outside surface of said prosthetic implant and a connection terminal connected with said electrode;
a surgical instrument having a gripping member for selectively gripping said prosthetic implant, wherein said gripping member includes at least one connector oriented to be connected with said connection terminal of said prosthetic implant when said gripping member is secured to said prosthetic implant; and
an electrical stimulation signal generator connected with said surgical instrument for providing electrical stimulation signals to said electrodes.

11. The arthroplasty system of claim 10, wherein said surgical instrument includes a stimulator switch for selectively providing said electrical stimulation signals to said electrodes.

12. The arthroplasty system of claim 10, wherein said connection terminal and said electrode are removably associated with said prosthetic implant.

13. The arthroplasty system of claim 10, further comprising a control unit for controlling said electrical stimulation signal generator.

14. The arthroplasty system of claim 10, wherein at least one surface of said prosthetic implant is coated with a bone growth material.

15. The arthroplasty system of claim 10, further comprising a control unit connected with a plurality of monitoring electrodes, wherein said monitoring electrodes are placed at predetermined locations of a human body for detecting a response to said electrical stimulation signals.

16. The arthroplasty system of claim 15, further comprising a display connected with said control unit, wherein said control unit is operable to generate a visual display associated with said response.

17. The arthroplasty system of claim 15, further comprising a speaker connected with said control unit, wherein said control unit is operable to generate an audible indication on said speaker as a function of said response.

18. The arthroplasty system of claim 10, wherein said connector includes a locking member that locks said connector in said connection terminal when said surgical instrument is secured to said prosthetic implant.

19. The arthroplasty system of claim 18, wherein said connection terminal and said electrode are removed from said prosthetic implant once said surgical instrument is disconnected from said prosthetic implant.

20. A surgical method for inserting an implant, comprising:
creating a window to a bone structure;
removing at least a portion of said bone structure;
providing a prosthetic implant having at least one electrode positioned on an outside surface of said prosthetic implant, wherein said electrode is connected with at least one connection terminal positioned in said prosthetic implant;
gripping said prosthetic implant with a surgical instrument such that a connector of said surgical instrument is electrically connected with said connection terminal of said prosthetic implant;
inserting said implant through said window to replace said portion of said bone structure that has been removed using said surgical instrument; and
generating an electrical stimulation signal that is selectively provided to said electrode while said prosthetic implant is being positioned to replace said portion of said bone structure.

21. The method of claim 20, wherein said prosthetic implant comprises a first articulate member and a second articulate member.

22. The method of claim 21, wherein said first articulate member includes a first bearing surface positioned in contact with an upper portion of said bone structure and said second articulate member includes a second bearing surface positioned in contact with a lower portion of said bone structure.

23. The method of claim 22, wherein said bone structure comprises adjacent vertebrae.

24. The method of claim 20, further comprising the step of removing said electrode and said connection terminal from said prosthetic implant once said prosthetic implant is inserted through said window to replace said portion of said bone structure that has been removed while leaving the remaining structure of said prosthetic implant in place.

25. The method of claim 20, wherein said surgical instrument includes a stimulator switch for selectively applying said electrical stimulation signal to said electrode while said prosthetic implant is being inserted in said window.

26. The method of claim 20, further comprising the step of placing a plurality of monitoring electrodes at predetermined locations on a human body.

27. The method of claim 26, further comprising the step of monitoring said plurality of monitoring electrodes with a control unit connected with said electrodes.

28. The method of claim 27, further comprising the step of generating an audible warning if a response to said electrical stimulation signal is received by one of said monitoring electrodes.

29. The method of claim 27, further comprising the step of generating a visual indication if a response to said electrical stimulation signal is received by one of said monitoring electrodes.

30. A spinal implant, comprising:
a first articulate member having a first electrode positioned on a first outside surface of said first articulate member and a first connection terminal connected with said first electrode;
a second articulate member having a second electrode positioned on a second outside surface of said first articulate member and a first connection terminal connected with said second electrode; and
wherein said first articulate member is pivotally connected with said second articulate member.
